SEQUENCE LISTING (20 sequences)

SEQ ID NO: 1 - DNA, 1128 bp, synthetic construct
  misc_feature 1..1128: note = Carboxylic ester hydrolase Arthrobacter globiformis esteraseQ44050 (Wild type)
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacagcgcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtttggtgaaaccagctgcttcggcaccgtttttatgaagagccacgcgcgtagcccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 2 - protein, 375 aa, synthetic construct
  REGION 1..375: note = Carboxylic ester hydrolase Arthrobacter globiformis esteraseQ44050 (Wild type)
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANSAVGDILDLPNLREVRAAGLSSAAGVASAEGMARVYAAALTGLAANGDRAAVAPLLSEETIQTVTAEQVFGIDRVFGETSCFGTVFMKSHARSPYGSYRAFGHDGASASLGFADPVYELAFGYVPQQAEPGGAGCRNLELSAAVRKAVTELAQ

SEQ ID NO: 3 - DNA, 1128 bp, synthetic construct
  misc_feature 1..1128: note = Arthrobacter globiformis esterase mutant S223M (variant 9)
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacatggcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtttggtgaaaccagctgcttcggcaccgtttttatgaagagccacgcgcgtagcccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 4 - protein, 375 aa, synthetic construct
  REGION 1..375: note = Arthrobacter globiformis esterase mutant S223M (variant 9)
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANMAVGDILDLPNLREVRAAGLSSAAGVASAEGMARVYAAALTGLAANGDRAAVAPLLSEETIQTVTAEQVFGIDRVFGETSCFGTVFMKSHARSPYGSYRAFGHDGASASLGFADPVYELAFGYVPQQAEPGGAGCRNLELSAAVRKAVTELAQ

SEQ ID NO: 5 - DNA, 1128 bp, synthetic construct
  misc_feature 1..1128: note = Arthrobacter globiformis esterase mutant S223L (variant 15)
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacttggcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtttggtgaaaccagctgcttcggcaccgtttttatgaagagccacgcgcgtagcccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 6 - protein, 375 aa, synthetic construct
  REGION 1..375: note = Arthrobacter globiformis esterase mutant S223L (variant 15)
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANLAVGDILDLPNLREVRAAGLSSAAGVASAEGMARVYAAALTGLAANGDRAAVAPLLSEETIQTVTAEQVFGIDRVFGETSCFGTVFMKSHARSPYGSYRAFGHDGASASLGFADPVYELAFGYVPQQAEPGGAGCRNLELSAAVRKAVTELAQ

SEQ ID NO: 7 - DNA, 1128 bp, synthetic construct
  misc_feature 1..1128: note = Arthrobacter globiformis esterase mutant S223F (variant 16)
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaactttgcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtttggtgaaaccagctgcttcggcaccgtttttatgaagagccacgcgcgtagcccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 8 - protein, 375 aa, synthetic construct
  REGION 1..375: note = Arthrobacter globiformis esterase mutant S223F (variant 16)
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANFAVGDILDLPNLREVRAAGLSSAAGVASAEGMARVYAAALTGLAANGDRAAVAPLLSEETIQTVTAEQVFGIDRVFGETSCFGTVFMKSHARSPYGSYRAFGHDGASASLGFADPVYELAFGYVPQQAEPGGAGCRNLELSAAVRKAVTELAQ

SEQ ID NO: 9 - DNA, 1128 bp, synthetic construct
  misc_feature 1..1128: note = Arthrobacter globiformis esterase mutant S315F (variant 18)
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacagcgcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtttggtgaaaccagctgcttcggcaccgtttttatgaagagccacgcgcgttttccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 10 - protein, 375 aa, synthetic construct
  REGION 1..375: note = Arthrobacter globiformis esterase mutant S315F (variant 18)
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANSAVGDILDLPNLREVRAAGLSSAAGVASAEGMARVYAAALTGLAANGDRAAVAPLLSEETIQTVTAEQVFGIDRVFGETSCFGTVFMKSHARFPYGSYRAFGHDGASASLGFADPVYELAFGYVPQQAEPGGAGCRNLELSAAVRKAVTELAQ

SEQ ID NO: 11 - DNA, 1128 bp, synthetic construct
  misc_feature 1..1128: note = Arthrobacter globiformis esterase mutant S315M (variant 19)
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacagcgcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtttggtgaaaccagctgcttcggcaccgtttttatgaagagccacgcgcgtatgccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 12 - protein, 375 aa, synthetic construct
  REGION 1..375: note = Arthrobacter globiformis esterase mutant S315M (variant 19)
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANSAVGDILDLPNLREVRAAGLSSAAGVASAEGMARVYAAALTGLAANGDRAAVAPLLSEETIQTVTAEQVFGIDRVFGETSCFGTVFMKSHARMPYGSYRAFGHDGASASLGFADPVYELAFGYVPQQAEPGGAGCRNLELSAAVRKAVTELAQ

SEQ ID NO: 13 - DNA, 1128 bp, synthetic construct
  misc_feature 1..1128: note = Arthrobacter globiformis esterase mutant F298W (variant 20)
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacagcgcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtggggtgaaaccagctgcttcggcaccgtttttatgaagagccacgcgcgtagcccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 14 - protein, 375 aa, synthetic construct
  REGION 1..375: note = Arthrobacter globiformis esterase mutant F298W (variant 20)
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANSAVGDILDLPNLREVRAAGLSSAAGVASAEGMARVYAAALTGLAANGDRAAVAPLLSEETIQTVTAEQVFGIDRVWGETSCFGTVFMKSHARSPYGSYRAFGHDGASASLGFADPVYELAFGYVPQQAEPGGAGCRNLELSAAVRKAVTELAQ

SEQ ID NO: 15 - DNA, 1128 bp, synthetic construct
  misc_feature 1..1128: note = Arthrobacter globiformis esterase double mutant S315M + S223M
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacatggcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtttggtgaaaccagctgcttcggcaccgtttttatgaagagccacgcgcgtatgccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 16 - protein, 375 aa, synthetic construct
  REGION 1..375: note = Arthrobacter globiformis esterase double mutant S315M + S223M
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANMAVGDILDLPNLREVRAAGLSSAAGVASAEGMARVYAAALTGLAANGDRAAVAPLLSEETIQTVTAEQVFGIDRVFGETSCFGTVFMKSHARMPYGSYRAFGHDGASASLGFADPVYELAFGYVPQQAEPGGAGCRNLELSAAVRKAVTELAQ

SEQ ID NO: 17 - DNA, 1128 bp, synthetic construct
  misc_feature 1..1128: note = Arthrobacter globiformis esterase double mutant S315F + S223M
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacatggcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcggtggcgccgctgctgagcgaggaaaccatccagaccgtgaccgcggagcaagttttcggcattgatcgtgtgtttggtgaaaccagctgcttcggcaccgtttttatgaagagccacgcgcgttttccgtacggtagctatcgtgcgttcggtcatgatggtgcgagcgcgagcctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 18 - protein, 375 aa, synthetic construct
  REGION 1..375: note = Arthrobacter globiformis esterase double mutant S315F + S223M
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANMAVGDILDLPNLREVRAAGLSSAAGVASAEGMARVYAAALTGLAANGDRAAVAPLLSEETIQTVTAEQVFGIDRVFGETSCFGTVFMKSHARFPYGSYRAFGHDGASASLGFADPVYELAFGYVPQQAEPGGAGCRNLELSAAVRKAVTELAQ

SEQ ID NO: 19 - DNA, 1128 bp, synthetic construct
  misc_feature 1..1128: note = Arthrobacter globiformis esterase triple mutant S315M + V274L +S331C
  source 1..1128: mol_type = other DNA
atggacgcgcagaccattgcgccgggttttgagagcgtggcggaactgttcggccgttttctgagcgaggatcgtgaatacagcgcgcaactggcggcgtatcaccgtggtgtgaaggttctggacatcagcggtggcccgcaccgtcgtccggatagcgtgaccggcgtttttagctgcagcaaaggtgtgagcggcctggttattgcgctgctggttcaggacggtttcctggacctggatgcggaagtggttaagtactggccggagtttggtgcggaaggcaaagcgaccattaccgtggcgcagctgctgagccaccaagcgggtctgctgggcgttgaaggtggcctgaccctggcggagtacaacaacagcgaactggctgcggcgaagctggcgcaaatgcgtccgctgtggaaaccgggtaccgcgttcggctatcacgcgctgaccatcggtgtgtttatggaggaactgtgccgtcgtattaccggcagcaccctgcaggagatctacgaacaacgtattcgtagcgttaccggtgcgcacttctttctgggtctgccggagagcgaggaaccgcgttatgcgaccctgcgttgggcggcggacccgagccagccgtggattgatccggcgagccactttggtctgagcgcgaacagcgcggtgggtgacattctggatctgccgaacctgcgtgaggttcgtgcggcgggtctgagcagcgcggcgggcgtggcgagcgcggagggtatggcgcgtgtttatgctgcggcgctgaccggtctggcggcgaacggtgaccgtgcggcgcttgcgccgctgctgagcgaggaaaccatccagaccgtgaccgcggagcaagttttcggcattgatcgtgtgtttggtgaaaccagctgcttcggcaccgtttttatgaagagccacgcgcgtatgccgtacggtagctatcgtgcgttcggtcatgatggtgcgagcgcgtgtctgggtttcgcggatccggtgtacgagctggcgtttggctatgtgccgcagcaagcggagccgggtggcgcgggttgccgtaacctggaactgagcgcggcggtgcgtaaagcggttaccgaactggcgcaatga

SEQ ID NO: 20 - protein, 375 aa, synthetic construct
  REGION 1..375: note = Arthrobacter globiformis esterase triple mutant S315M + V274L +S331C
MDAQTIAPGFESVAELFGRFLSEDREYSAQLAAYHRGVKVLDISGGPHRRPDSVTGVFSCSKGVSGLVIALLVQDGFLDLDAEVVKYWPEFGAEGKATITVAQLLSHQAGLLGVEGGLTLAEYNNSELAAAKLAQMRPLWKPGTAFGYHALTIGVFMEELCRRITGSTLQEIYEQRIRSVTGAHFFLGLPESEEPRYATLRWAADPSQPWIDPASHFGLSANSAVGDILDLPNLREVRAAGLSSAAGVASAEGMARVYAAALTGLAANGDRAALAPLLSEETIQTVTAEQVFGIDRVFGETSCFGTVFMKSHARMPYGSYRAFGHDGASACLGFADPVYELAFGYVPQQAEPGGAGCRNLELSAAVRKAVTELAQ

(19) United States
(12) Patent Application Publication
(10) Pub. No.: US 2026/0286323 A1
(43) Pub. Date: Sep. 24, 2026

(54) Novel Esterase Mutants And Uses Thereof As Stereoselective Catalyst

(71) Applicant: Endura S.P.A., Bologna (IT)

(72) Inventors: Roberto VERGA, Villanova del Sillaro (IT); Simone STASI, Villanova del Sillaro (IT); Silvia RAPACIOLI, Villanova del Sillaro (IT); Carla MARCHIORO, Villafranca di Verona (VR) (IT); Silvia CARLONI, Ravenna (IT); Leni MORONI, Ravenna (IT); Valerio BORZATTA, Bologna (IT)

(21) Appl. No.: 18/998,398

(22) PCT Filed: Jul. 26, 2022
(86) PCT No.: PCT/EP2022/070973

§ 371 Date: Jan. 24, 2025

(51) Int. Cl. C12N 9/18; C12P 7/40; C12P 41/00

(52) U.S. Cl. CPC C12N 9/18; C12P 7/40; C12P 41/005; C12Y 301/01

(57) ABSTRACT

The present invention concerns the field of enzyme mutants, and in particular Arthrobacter globiformis esterase mutants suitable for asymmetrical hydrolysis of the cyclopropane derivatives, specifically chrysanthemate ester in any combination of the four stereoisomers in order to selectively obtain (1R,3R)-chrysanthemic acid or a salt thereof. The invention further describes an expression vector including the Arthrobacter globiformis esterase mutant and transformed host microorganisms harboring the vectors. The present invention further relates to the use of Arthrobacter globiformis esterase mutants to selectively obtain (1R,3R)-chrysanthemic acid. Under a further aspect, the invention relates to the use of Arthrobacter globiformis esterase mutants for the asymmetrical hydrolysis of cyclopropane racemate derivatives having at least two chiral stereocenters, preferably for the asymmetrical hydrolysis of cyclopropane racemate derivatives having at least two chiral stereocenters.

### FIELD OF THE INVENTION

The present invention concerns the field of enzyme mutants, and in particular Arthrobacter globiformis esterase mutants suitable for asymmetrically hydrolyzing the cyclopropane derivatives, specifically chrysanthemate ester in any combination of the four stereoisomers in order to selectively obtain (1R,3R)-chrysanthemic acid or a salt thereof.

The invention further describes an expression vector including the Arthrobacter globiformis esterase mutant and transformed host microorganisms harboring the vectors.

The present invention further relates to the use of Arthrobacter globiformis esterase mutants to selectively obtain (1R,3R)-chrysanthemic acid. Under a further aspect, the invention relates to the use of Arthrobacter globiformis esterase mutants for the asymmetrical hydrolysis of cyclopropane racemate derivatives having at least two chiral stereocenters, preferably for the asymmetrical hydrolysis of cyclopropane racemate derivatives having at least two chiral stereocenters.

### STATE OF THE ART

Tanacetum cinerariifolium or Dalmatian chrysanthemum is a species of white flowering plant in the aster family, which was formerly part of the genus Pyrethrum, but now placed in the genus Chrysanthemum.

This plant is a natural source of “pyrethrum” insecticides, thereby giving it a great economic importance. The flowers are pulverized and the active components, called pyrethrins, contained in the seed cases, are extracted and sold in the form of an oleoresin which can be applied as a suspension in water or oil, or as a powder.

Pyrethrins are a mixture of six structurally related insecticidal esters formed by a combination of two acids (chrysanthemic acid and pyrethric acid) and different alcohols. The esters of chrysanthemic acid are called pyrethrin I, cinerin I, and jasmolin I, respectively, and are together known as pyrethrins I, whereas the esters of pyrethric acid are called pyrethrin II, cinerin II, and jasmolin II, and together, the pyrethrins II Pyrethrins attack the nervous systems of all insects, inhibiting female mosquitoes from biting. When present in amounts less than those fatal to insects, they still appear to have an insect repellent effect.

They are harmful to fish but are far less toxic to mammals and birds than many synthetic insecticides and are not persistent, being biodegradable and also decompose easily on exposure to light. They are considered to be amongst the safest insecticides for use around food.

After the chemical structure of “natural pyrethrins” was elucidated, useful synthetic pyrethroids provided with various characteristics have been developed, leading to the advancement of pyrethroid chemistry.

The Chrysanthemic acid is the peculiar intermediate that is related to a variety of natural and synthetic insecticides. In particular, the pyrethroids are derivatives of the chrysanthemic acid that can be found in different possible 4 stereoisomers i.e. (1R,3R) or (1R,3S) or (1S,3R) or (1S,3S).

The (1R,3R)- or (+)-trans-chrysanthemic acid (one of the four stereoisomers) is generally considered as the most interesting and effective of the four stereoisomers:

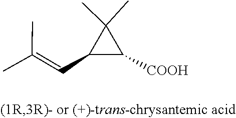

Specifically, as also reported in Nishizawa et al. (“Stereoselective Production of (+)-trans-Chrysanthemic Acid by a Microbial Esterase: Cloning, Nucleotide Sequence, and Overexpression of the Esterase Gene of Arthrobacter globiformis in Escherichia coli; Appl Environ Microbiol. 1995; 61:3208-15), a highly stereoselective enzymatic production of the (+)-trans-chrysanthemic acid would have a great advantage over the conventional chemical processes to selectively obtain the target isomer. The Esterase Gene of Arthrobacter globiformis was characterized and reported to be highly stereospecific, although difficult to use in large scale production of insecticides, due to product inhibition.

It is therefore object of the present invention the development of a stereoselective hydrolysis of the chrysanthemic esters.

It is known that the chrysanthemic acid can be obtained by hydrolysis of the appropriate chrysanthemic esters, in particular of ethyl chrysanthemate in racemic form.

The ethyl chrysanthemate (or related esters) can be found in four isomers:

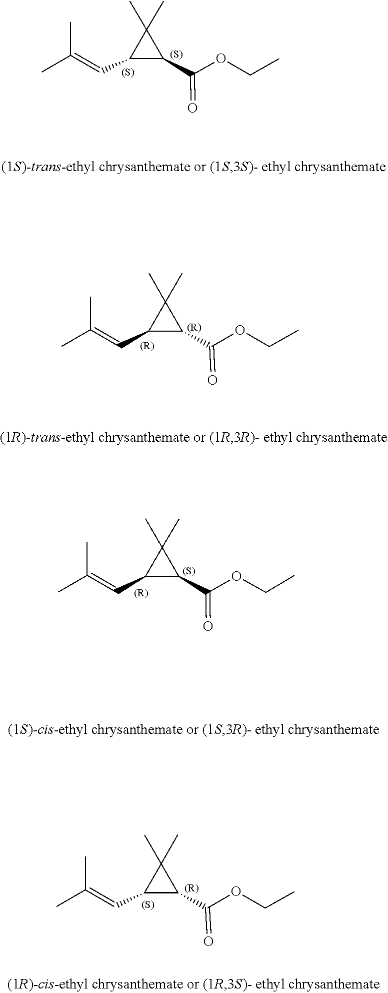

The stereoisomer mixture is the starting material for producing the chrysanthemic acid.

A further object of the present invention is hence to selectively produce the (1R,3R) chrysanthemic acid from the ethyl chrysanthemate racemate.

### SUMMARY OF THE INVENTION

The inventors surprisingly found a new esterase capable of selectively hydrolyzing an ester racemate of chrysanthemic acid to selectively produce (1R,3R) chrysanthemic acid or salts thereof.

The present invention hence concerns an Arthrobacter globiformis esterase mutant having a sequence comprising mutations of amino acid residue S at position 315, amino acid residue S at position 223 or amino acid residue F at position 298 of SEQ ID NO: 2.

Under a second aspect, the present invention regards an expression vector including an Arthrobacter globiformis esterase mutant nucleotide sequence chosen from the group consisting of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

A third aspect of the present invention relates to a transformed host microorganism containing the expression vector, said vector including an Arthrobacter globiformis esterase mutant nucleotide sequence chosen from the group consisting of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

In a fourth aspect, the present invention relates to the use of an Arthrobacter globiformis esterase mutant according to the invention, for the selective preparation of (1R,3R)-chrysanthemic acid.

In a preferred aspect the invention relates to the Arthrobacter globiformis esterase mutant being capable of asymmetrically hydrolyzing cyclopropane racemate derivatives having at least two chiral stereocenters.

Therefore, in a fifth aspect the invention relates to the use of the Arthrobacter globiformis esterase mutant for the asymmetrical hydrolysis of cyclopropane racemate derivatives having at least two chiral stereocenters.

In a preferred and advantageous aspect the invention relates to the Arthrobacter globiformis esterase mutant being capable of asymmetrically hydrolyzing (C₁-C₆)alkyl chrysanthemate, preferably ethyl chrysanthemate.

Therefore, in a sixth aspect the invention relates to the use of the Arthrobacter globiformis esterase mutant for asymmetrically hydrolyzing (C₁-C₆)alkyl chrysanthemate, preferably ethyl chrysanthemate.

As will be further described in the detailed description of the invention, the solution underlying the present invention is that of making available an innovative esterase with improved properties in obtaining the desired (1R,3R) chrysanthemic acid for the production of pyrethroid insecticides.

The problem of providing (1R,3R)-chrysanthemic acid is solved by the present finding, particularly by the identification of mutants of the Arthrobacter globiformis esterase, having an improved enzyme kinetic, an improved enzyme stability and/or a reduced product inhibition when compared to the wild-type Arthrobacter globiformis esterase of SEQ ID NO:2, as identified in the attached claims.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-10, wherein:

FIG. 1: Comparison of the specific activity of raw extracts of cells expressing different esterase enzyme variants.

The activity, expressed as a percentage of the specific activity of the wild-type enzyme, was measured using 10% (v/v) racemate of ethyl ester of chrysanthemic acid as a substrate, measuring (1R,3R) chrysanthemic acid formation by HPLC analysis.

FIG. 2. Schematic representation of the functioning of the gene library generation kit: 1. Megaprimer mutants synthesis: amplification of the gene encoding for the esterase S315M using a DNA polymerase prone to insertion mutations allows to obtain the gene variants which are purified. 2. Vector Libraries containing mutated genes generated in 1. are used as a megaprimer to amplify the target vector using a high-fidelity DNA Polymerase to obtain a library of vectors containing mutated gene versions of the S315M esterase enzyme.

FIG. 3: Representative graph of the variation of the specific activity as a function of the product concentration per an enzyme affected by product inhibition.

FIG. 4: Schematic representation of the selection scheme of the enzymatic variants obtained through random mutagenesis.

FIG. 5. Percentage increase in the residual activity of the 61 selected variants compared to the starting enzyme S315M.

FIG. 6. Percentage increase of the specific activity and hence of the inhibition in the presence of (1R,3R) chrysanthemic acid, of the 61 variants selected with respect to the starting enzyme S315M.

FIG. 7. Percentage increase in the residual activity of the 25 selected variants compared to the starting enzyme S315M.

FIG. 8. Chromatograms, obtained by HPLC analysis, relating to 20 hours of reaction conducted with the enzyme S315M and the mutated variants V274L-S315M-S331C (triple mutant), S315F, S223M.

FIG. 9. Calibration line of (1R,3R)-chrysanthemic acid: (1R,3R)-chrysanthemic acid amount (g/L) in pH stat and % yield of bioconversion for the enzymatic variants of the present invention and for the S315M enzyme used as a starting point for random mutagenesis.

### DETAILED DESCRIPTION OF THE INVENTION

The present invention hence concerns an Arthrobacter globiformis esterase mutant having a sequence comprising mutations of amino acid residue S at position 315, amino acid residue S at position 223 or amino acid residue F at position 298 of SEQ ID NO: 2.

Without being bound to any theory the inventors deem that the Arthrobacter globiformis esterase mutants herein claimed allow to obtain a higher yield in the selective conversion of cyclopropane derivatives having at least two chiral centers, preferably the racemate of (C₁-C₆) alkyl ester of chrysanthemic acid to (1R,3R)-chrysanthemic acid or a salt thereof, more preferably the racemate of ethyl ester of chrysanthemic acid:

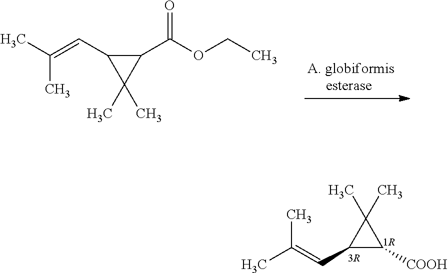

by influencing the following three aspects of the catalysis:
i) improved enzyme stability at different temperatures and pH values: the enzyme has good operational stability under basic conditions, in the pH range between 9.0 and 11.00. The best conversion yields are also obtained in this range, preferably in the 10.0-10.5 range, with significant decreases below 8.5-9.0;
ii) improved kinetic parameters; and
iii) reduction of product inhibition, when compared to the wild-type Arthrobacter globiformis esterase with amino acid sequence described in SEQ ID NO:2. The mutants described have undergone improved changes in catalytic performance, achieving conversion yields of up to 88-95% compared to what is achievable with WT enzyme with amino acid sequence reported in SEQ ID NO:2, which does not exceed values of 40-49%, with pH-stat during the reaction. The mutations have increased the affinity towards the substrate, without altering the high stereospecificity of the enzyme and decreasing the product inhibition that remains evident in WT enzyme.

For the purposes of the present invention, the term:
“(1R,3R)-chrysanthemic acid” as used herein is intended to be synonymous to “(1R)-trans-chrysanthemic acid”, “(+)-trans-chrysanthemic acid” and “(1R,3R)-2,2-Dimethyl-3-(2-methyl-1-propenyl)cyclopropane-1-carboxylic acid” and having the CAS number 4638-92-0;
“(1R,3R)-chrysanthemic acid or its salts” means a salt of a metal selected from alkali metals and earth alkali metals such as potassium, sodium, calcium, magnesium, etc.;
“cyclopropane derivatives having at least two chiral centers” means cyclopropane compounds having at least two asymmetric carbon atoms so as to have at least two stereoisomers, preferably four stereoisomers;
“(C₁-C₆)alkyl esters of chrysanthemic acid” means esters of a linear or branched alkyl chain of 1 to 6 carbon atoms of chrysanthemic acid, such as methyl, ethyl, propyl, isopropyl, butyl, isobutyl, sec-butyl, tert-butyl, pentyl, iso-pentyl, hexyl, etc.;
“Ethyl ester of chrysanthemic acid” as used herein is intended to be synonymous to “ethyl 2,2-dimethyl-3-(2-methylprop-1-enyl)cyclopropane-1-carboxylate” and has the CAS number 97-41-6. According to the present invention, the ethyl ester of chrysanthemic acid can be provided in any ratio of trans/cis forms, for example from 60/40 to 100/0, preferably 65/35, 70/30, 80/20, 90/10, 92/8, 98/2.

In a preferred aspect, in the Arthrobacter globiformis esterase mutant according to the present invention said amino acid residue S at position 315, said amino acid residue S at position 223 and said amino acid residue F at position 298 of SEQ ID NO: 2 are replaced with a non-polar amino acid residue chosen from the group consisting of M, F, L, W, A, I, P or V. It was surprisingly seen by the inventors that by replacing the amino acid residues present in position 315, 223 or 298 of SEQ ID NO:2 with a non-polar amino acid, the characteristics of the esterase enzyme are improved and maintains the capability of asymmetrically hydrolyzing racemic esters of chrysanthemic acid.

For the purposes of the present invention, amino acid residues are indicated by their “one letter code”, wherein “M” corresponds to Met or methionine, “F” corresponds to Phe or phenylalanine, “S” corresponds to Ser or serine and so on, as a person with ordinary skill in the art would understand.

In a more preferred aspect, in the Arthrobacter globiformis esterase mutant of the present invention, said amino acid residue S at position 315, said amino acid residue S at position 223 and said amino acid residue F at position 298 of SEQ ID NO: 2 are replaced with a non-polar amino acid residue chosen from the group consisting of M, F, L or W.

In a still more preferred aspect, the Arthrobacter globiformis esterase mutant of the present invention, has one of the following mutations S315M, S315F, S223M, S223L, S223F or F298W in the amino acid sequence of SEQ ID NO: 2.

In a still more preferred aspect, the Arthrobacter globiformis esterase mutant of the present invention, has one further mutation (double mutant) or two further mutations (triple mutant), and is preferably chosen from the group consisting of:
the double mutant having the mutations S315M and S223M in the amino acid sequence of SEQ ID NO: 2;
the double mutant having the mutations S315F and S223M in the amino acid sequence of SEQ ID NO: 2; and
the triple mutant having the mutations S315M, V274L and S331C in the amino acid sequence of SEQ ID NO: 2.

Preferred Arthrobacter globiformis esterase mutants have the following amino acid sequences: SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 or SEQ ID NO: 20.

More preferably the Arthrobacter globiformis esterase mutant according to the present invention has the amino acid sequence of SEQ ID NO: 4, SEQ ID NO: 10 or SEQ ID NO: 12. Still more preferably the Arthrobacter globiformis esterase mutant according to the present invention has the amino acid sequence of SEQ ID NO: 12 and a corresponding nucleotide sequence such as SEQ ID NO:11. This mutant is the S315M mutant of the Arthrobacter globiformis esterase.

The invention also provides for the nucleotide sequences of the Arthrobacter globiformis esterase mutants, the mutants have the nucleotide sequences described in SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

Under a second aspect, the present invention regards an expression vector including an Arthrobacter globiformis esterase mutant nucleotide sequence chosen from the group consisting of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

A third aspect of the present invention relates to a transformed host microorganism containing the expression vector, said vector including an Arthrobacter globiformis esterase mutant nucleotide sequence chosen from the group consisting of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

In a fourth aspect, the present invention relates to the use of an Arthrobacter globiformis esterase mutant according to the invention, for the selective preparation of (1R,3R)-chrysanthemic acid or a salt thereof.

In a preferred aspect the invention relates to the Arthrobacter globiformis esterase mutant being capable of asymmetrically hydrolyzing cyclopropane racemate derivatives having at least two chiral stereocenters.

Therefore, in a fifth aspect the invention relates to the use of the Arthrobacter globiformis esterase mutant for the asymmetrical hydrolysis of cyclopropane racemate derivatives having at least two chiral stereocenters.

In a preferred and advantageous aspect, the invention relates to the Arthrobacter globiformis esterase mutant being capable of asymmetrically hydrolyzing (C₁-C₆)alkyl esters of chrysanthemic acid, preferably ethyl ester of chrysanthemic acid.

Therefore, in a sixth aspect the invention relates to the use of the Arthrobacter globiformis esterase mutant for asymmetrically hydrolyzing (C₁-C₆)alkyl ester of chrysanthemic acid, preferably ethyl ester of chrysanthemic acid.

For the purposes of the present disclosure, each Arthrobacter globiformis esterase sequence (wild type and mutant) has a corresponding SEQ ID NO: as follows, wherein for each mutant sequence the mutated nucleotide triplet or amino acid is indicated in bold and underlined:

SEQ ID NO: 1 corresponds to the nucleotide sequence of:
Carboxylic ester hydrolase Arthrobacter globiformis esterase Q44050 (Wild type)
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACAGCGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTAGCCCGTACGGTAGCTATCGTGCGTTCGGTCATGATGGTGCGAGCGCG
AGCCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 2 corresponds to the amino acid sequence of:
Carboxylic ester hydrolase Arthrobacter globiformis esterase Q44050 (Wild type)
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANSAVGDILD
LPNLREVRAAGLSSAAGVASAEGMARVYAAALTGLAANGDRAAVAPLLSEETIQTV
TAEQVFGIDRVFGETSCFGTVFMKSHARSPYGSYRAFGHDGASASLGFADPVYELAF
GYVPQQAEPGGAGCRNLELSAAVRKAVTELAQ
SEQ ID NO: 3 corresponds to the nucleotide sequence of:
Arthrobacter globiformis esterase mutant S223M (variant 9)
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACATGGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTAGCCCGTACGGTAGCTATCGTGCGTTCGGTCATGATGGTGCGAGCGCG
AGCCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 4 corresponds to the amino acid sequence of:
Arthrobacter globiformis esterase mutant S223M (variant 9)
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANMAVGDILD
LPNLREVRAAGLSSAAGVASAEGMARVYAAALTGLAANGDRAAVAPLLSEETIQTV
TAEQVFGIDRVFGETSCFGTVFMKSHARSPYGSYRAFGHDGASASLGFADPVYELAF
GYVPQQAEPGGAGCRNLELSAAVRKAVTELAQ
SEQ ID NO: 5 corresponds to the nucleotide sequence of:
Arthrobacter globiformis esterase mutant S223L (variant 15)
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACTTGGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTAGCCCGTACGGTAGCTATCGTGCGTTCGGTCATGATGGTGCGAGCGCG
AGCCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 6 corresponds to the amino acid sequence of:
Arthrobacter globiformis esterase mutant S223L (variant 15)
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANLAVGDILD
LPNLREVRAAGLSSAAGVASAEGMARVYAAALTGLAANGDRAAVAPLLSEETIQTV
TAEQVFGIDRVFGETSCFGTVFMKSHARSPYGSYRAFGHDGASASLGFADPVYELAF
GYVPQQAEPGGAGCRNLELSAAVRKAVTELAQ
SEQ ID NO: 7 corresponds to the nucleotide sequence of:
Arthrobacter globiformis esterase mutant S223F (variant 16)
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACTTTGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTAGCCCGTACGGTAGCTATCGTGCGTTCGGTCATGATGGTGCGAGCGCG
AGCCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 8 corresponds to the amino acid sequence of:
Arthrobacter globiformis esterase mutant S223F (variant 16)
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANFAVGDILD
LPNLREVRAAGLSSAAGVASAEGMARVYAAALTGLAANGDRAAVAPLLSEETIQTV
TAEQVFGIDRVFGETSCFGTVFMKSHARSPYGSYRAFGHDGASASLGFADPVYELAF
GYVPQQAEPGGAGCRNLELSAAVRKAVTELAQ
SEQ ID NO: 9 corresponds to the nucleotide sequence of:
Arthrobacter globiformis esterase mutant S315F (variant 18)
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACAGCGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTTTTCCGTACGGTAGCTATCGTGCGTTCGGTCATGATGGTGCGAGCGCG
AGCCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 10 corresponds to the amino acid sequence of:
Arthrobacter globiformis esterase mutant S315F (variant 18)
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANSAVGDILD
LPNLREVRAAGLSSAAGVASAEGMARVYAAALTGLAANGDRAAVAPLLSEETIQTV
TAEQVFGIDRVFGETSCFGTVFMKSHARFPYGSYRAFGHDGASASLGFADPVYELAF
GYVPQQAEPGGAGCRNLELSAAVRKAVTELAQ
SEQ ID NO: 11 corresponds to the nucleotide sequence of:
Arthrobacter globiformis esterase mutant S315M (variant 19)
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACAGCGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTATGCCGTACGGTAGCTATCGTGCGTTCGGTCATGATGGTGCGAGCGCG
AGCCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 12 corresponds to the amino acid sequence of:
Arthrobacter globiformis esterase mutant S315M (variant 19)
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANSAVGDILD
LPNLREVRAAGLSSAAGVASAEGMARVYAAALTGLAANGDRAAVAPLLSEETIQTV
TAEQVFGIDRVFGETSCFGTVFMKSHARMPYGSYRAFGHDGASASLGFADPVYELAF
GYVPQQAEPGGAGCRNLELSAAVRKAVTELAQ
SEQ ID NO: 13 corresponds to the nucleotide sequence of:
Arthrobacter globiformis esterase mutant F298W (variant 20)
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACAGCGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTGGGGTGAAACCAGCTGCTTCGGCACCGTTTTTATGAAGAGCC
ACGCGCGTAGCCCGTACGGTAGCTATCGTGCGTTCGGTCATGATGGTGCGAGCGC
GAGCCTGGGTTTCGCGGATCCGGTGTACGAGCTGGCGTTTGGCTATGTGCCGCAG
CAAGCGGAGCCGGGTGGCGCGGGTTGCCGTAACCTGGAACTGAGCGCGGCGGTG
CGTAAAGCGGTTACCGAACTGGCGCAATGA
SEQ ID NO: 14 corresponds to the amino acid sequence of:
Arthrobacter globiformis esterase mutant F298W (variant 20)
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANSAVGDILD
LPNLREVRAAGLSSAAGVASAEGMARVYAAALTGLAANGDRAAVAPLLSEETIQTV
TAEQVFGIDRVWGETSCFGTVFMKSHARSPYGSYRAFGHDGASASLGFADPVYELAF
GYVPQQAEPGGAGCRNLELSAAVRKAVTELAQ
SEQ ID NO: 15 corresponds to the nucleotide sequence of:
Arthrobacter globiformis esterase double mutant S315M + S223M
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACATGGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTATGCCGTACGGTAGCTATCGTGCGTTCGGTCATGATGGTGCGAGCGCG
AGCCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 16 corresponds to the amino acid sequence of:
Arthrobacter globiformis esterase double mutant S315M + S223M
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANMAVGDILD
LPNLREVRAAGLSSAAGVASAEGMARVYAAALTGLAANGDRAAVAPLLSEETIQTV
TAEQVFGIDRVFGETSCFGTVFMKSHARMPYGSYRAFGHDGASASLGFADPVYELAF
GYVPQQAEPGGAGCRNLELSAAVRKAVTELAQ
SEQ ID NO: 17 corresponds to the nucleotide sequence of:
Arthrobacter globiformis esterase double mutant S315F + S223M
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACATGGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGGTGGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTTTTCCGTACGGTAGCTATCGTGCGTTCGGTCATGATGGTGCGAGCGCG
AGCCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 18 corresponds to the amino acid sequence of:
Arthrobacter globiformis esterase double mutant S315F + S223M
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANMAVGDILD
LPNLREVRAAGLSSAAGVASAEGMARVYAAALTGLAANGDRAAVAPLLSEETIQTV
TAEQVFGIDRVFGETSCFGTVFMKSHARFPYGSYRAFGHDGASASLGFADPVYELAF
GYVPQQAEPGGAGCRNLELSAAVRKAVTELAQ
SEQ ID NO: 19 corresponds to the nucleotide sequence of:
Arthrobacter globiformis esterase triple mutant S315M + V274L + S331C
ATGGACGCGCAGACCATTGCGCCGGGTTTTGAGAGCGTGGCGGAACTGTTCGGCC
GTTTTCTGAGCGAGGATCGTGAATACAGCGCGCAACTGGCGGCGTATCACCGTGG
TGTGAAGGTTCTGGACATCAGCGGTGGCCCGCACCGTCGTCCGGATAGCGTGACC
GGCGTTTTTAGCTGCAGCAAAGGTGTGAGCGGCCTGGTTATTGCGCTGCTGGTTC
AGGACGGTTTCCTGGACCTGGATGCGGAAGTGGTTAAGTACTGGCCGGAGTTTGG
TGCGGAAGGCAAAGCGACCATTACCGTGGCGCAGCTGCTGAGCCACCAAGCGGG
TCTGCTGGGCGTTGAAGGTGGCCTGACCCTGGCGGAGTACAACAACAGCGAACTG
GCTGCGGCGAAGCTGGCGCAAATGCGTCCGCTGTGGAAACCGGGTACCGCGTTCG
GCTATCACGCGCTGACCATCGGTGTGTTTATGGAGGAACTGTGCCGTCGTATTACC
GGCAGCACCCTGCAGGAGATCTACGAACAACGTATTCGTAGCGTTACCGGTGCGC
ACTTCTTTCTGGGTCTGCCGGAGAGCGAGGAACCGCGTTATGCGACCCTGCGTTG
GGCGGCGGACCCGAGCCAGCCGTGGATTGATCCGGCGAGCCACTTTGGTCTGAGC
GCGAACAGCGCGGTGGGTGACATTCTGGATCTGCCGAACCTGCGTGAGGTTCGTG
CGGCGGGTCTGAGCAGCGCGGCGGGCGTGGCGAGCGCGGAGGGTATGGCGCGTG
TTTATGCTGCGGCGCTGACCGGTCTGGCGGCGAACGGTGACCGTGCGGCGCTTGC
GCCGCTGCTGAGCGAGGAAACCATCCAGACCGTGACCGCGGAGCAAGTTTTCGGC
ATTGATCGTGTGTTTGGTGAAACCAGCTGCTTCGGCACCGTTTTTATGAAGAGCCA
CGCGCGTATGCCGTACGGTAGCTATCGTGCGTTCGGTCATGATGGTGCGAGCGCG
TGTCTGGGTTTCGCGGATCCGGTGTACGAGCTGGCGTTTGGCTATGTGCCGCAGC
AAGCGGAGCCGGGTGGCGCGGGTTGCCGTAACCTGGAACTGAGCGCGGCGGTGC
GTAAAGCGGTTACCGAACTGGCGCAATGA
SEQ ID NO: 20 corresponds to the amino acid sequence of:
Arthrobacter globiformis esterase triple mutant S315M + V274L + S331C
MDAQTIAPGFESVAELFGRFLSEDREYSAQLAAYHRGVKVLDISGGPHRRPDSVTGVF
SCSKGVSGLVIALLVQDGFLDLDAEVVKYWPEFGAEGKATITVAQLLSHQAGLLGVE
GGLTLAEYNNSELAAAKLAQMRPLWKPGTAFGYHALTIGVFMEELCRRITGSTLQEI
YEQRIRSVTGAHFFLGLPESEEPRYATLRWAADPSQPWIDPASHFGLSANSAVGDILD
LPNLREVRAAGLSSAAGVASAEGMARVYAAALTGLAANGDRAALAPLLSEETIQTV
TAEQVFGIDRVFGETSCFGTVFMKSHARMPYGSYRAFGHDGASACLGFADPVYELAF
GYVPQQAEPGGAGCRNLELSAAVRKAVTELAQ

Various embodiments and aspects of the present invention as delineated hereinabove and as claimed in the claims section below find experimental support in the following examples.

### Examples

Reference is now made to the following examples, which together with the above descriptions illustrate some embodiments of the invention.

### Example 1. Generation of a Gene Library of Mutants Encoding for the Esterase Enzyme

1045 variants of the esterase enzyme were generated by random mutagenesis, in order to select enzyme variants with a decreased product inhibition by High Throughput Screening (HTS). Specifically, the most promising mutant, the S315M mutant (also referred to as variant 19) was obtained.

In particular the S315M esterase mutant is characterized by a higher activity on the ester of chrysanthemic acid substrate and is used as a starting point (template) for a random mutagenesis cycle, as here described.

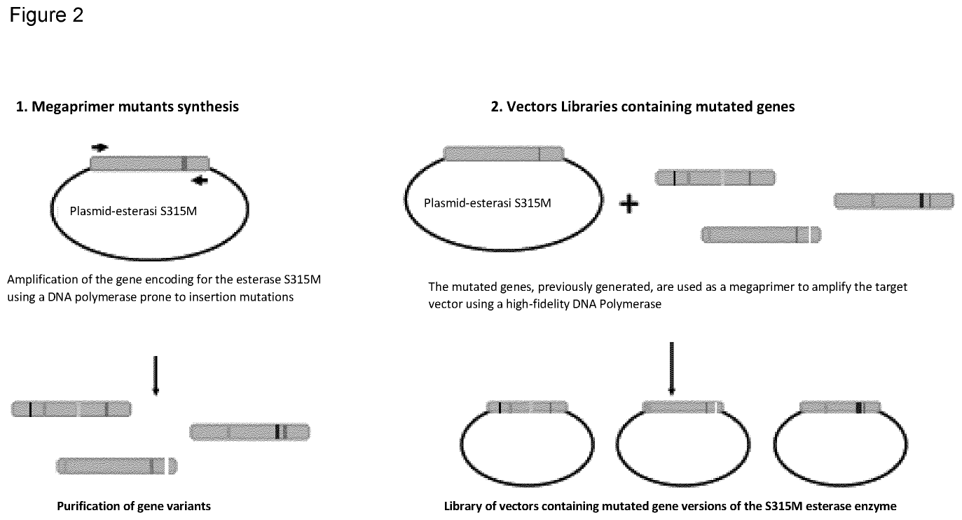

The gene library was generated by random mutagenesis performed by using the commercial kit Genemorph II Random Mutagenesis Kit (Agilent cat. N° 200550), using the S315M esterase gene as a template (FIG. 2).

As described in FIG. 2, the method is divided into 2 phases:
1. The gene encoding the S315M esterase enzyme is used as a template in a PCR reaction conducted using the error-prone Mutazyme II DNA polymerase, and an amount of DNA and amplification cycles such as to determine a low mutation frequency, i.e. 0-4.5 mutations/Kbase. The gene variants thus obtained were loaded onto gel and the corresponding band was excised and then purified.
2. The gene variants of the S315M enzyme, generated in phase 1, were used as megaprimer for the amplification of the target vector (generically a vector with a T7 promoter) by means of a high-fidelity DNA polymerase to avoid the introduction of mutations in the backbone of the vector. The mutated vector library was then purified and subsequently used for the transformation of E. coli cells.

### Example 2. Gene Library Expression in Overproducing E. coli Cells

The “Esterase mutant” vector library was first fully amplified in E. coli DH5a. Following the amplification, plasmid DNA extraction was carried out as well as the transformation of overexpressing E. coli cells, suitable for gene expression.

The cells thus transformed were plated on selective medium obtaining 10,832 colonies, after incubation over-night at 37° C.

Of these, 1045 were randomly selected to then proceeded to the gene expression and subsequently to HTS.

Each colony was inoculated into a deep well in 1 mL of ZYM5052 medium containing lactose, which is able to induce the expression of the genes encoding the variants of the esterase enzyme, inserted in the vector.

After an incubation of 24 hours at 37° C. the cells were collected by centrifugation and the pellet was processed for subsequent analyses.

### Example 3. High Throughput Screening (HTS) for the Selection of Enzymatic Variants with Decreased Product Inhibition

In enzymatic reactions the enzymes may be subject to inhibition by the substrate or by the product.

Substrate inhibition: In this case, there is a decrease in the initial specific activity as a function of substrate concentration.

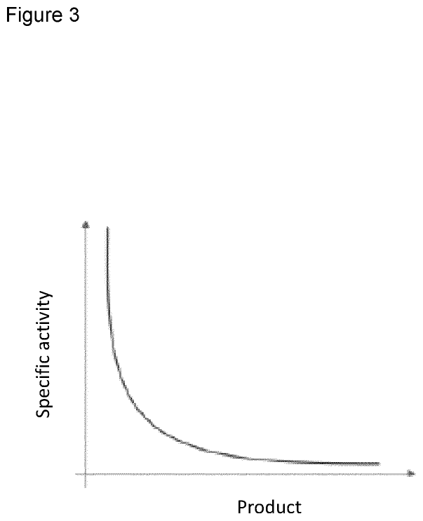

Product inhibition: In this case, the specific activity decreases until it becomes zero only when a certain concentration of product is accumulated, FIG. 3 is representative of the phenomenon. As a direct consequence, an incomplete bioconversion of the substrate into the product could occur.

Specifically, as reported in Nishizawa et al. (“Stereoselective Production of (+)-trans-Chrysanthemic Acid by a Microbial Esterase: Cloning, Nucleotide Sequence, and Overexpression of the Esterase Gene of Arthrobacter globiformis in Escherichia coli; Appl Environ Microbiol. 1995; 61:3208-15), the esterase enzyme object of the present study is subject to product inhibition, therefore in the mutant screening the specific activity was evaluated both in the absence and in the presence of the product, (1R,3R)-chrysanthemic acid, providing the chromogenic substrate p-nitro-phenyl-butyrate. The concentration of (1R,3R) chrysanthemic acid tested is equal to 100 mM, value chosen as obtained at the end of the bioconversion conducted with the S315M enzyme.

The inhibition was then evaluated in terms of percentage of residual activity, or the percentage ratio between the specific activity with acid and the specific activity without acid. Consequently, the higher the percentage of residual activity, the lower the degree of inhibition.

### Experimental Protocol:

Previously collected cell pellets were lysed by resuspension in 200 μL of Cellytic™ (enzymatic cocktail in detergent) and incubation at room temperature for 2 hours in agitation. The obtained lysate was clarified by centrifugation and the supernatant, ie. the total crude extract was used, after dilution, for screening.

Screening was set up in 96-well multiplate and reactions were conducted at a temperature of 25° C. for 10 minutes in 100 mM sodium phosphate buffer providing the substrate chromogenic p-nitro-phenyl-butyrate at the final concentration of 4 mM, in the absence and presence of (1R,3R) chrysanthemic acid at the final concentration of 100 mM. The absorbance was monitored over time by acquiring readings at 405 nm every 16 seconds.

The enzymatic activity (U/mL) is therefore defined as:

$$\frac{ΔAbs / min}{ε}$$

where ε is the molar extinction coefficient of para-nitro-phenol.

In order to calculate the specific activity (U/mg) of the enzymatic variants in the presence or absence of the acid, the total proteins in the crude extracts were quantified through the Bradford method, using BSA as the reference protein for the calibration curve.

The HTS procedure allowed to obtain and select 25 enzyme variants.

### Example 4. Quantification of (1R, 3R) Chrysanthemic Acid by HPLC in 25 Selected Enzymatic Variants

The production of (1R,3R) chrysanthemic acid was evaluated for the 25 enzymatic variants selected by HTS to which chrysanthemic acid ethyl ester was added as a substrate. Specifically, the acid product was quantified by reverse-phase HPLC analysis, with a gradient elution of a solution composed of 50% Methanol 50% Acetonitrile and 0.1% TFA.

The quantification of (1R, 3R) chrysanthemic acid allowed for:
i) the calculation of the specific enzymatic activity (U/mg) on chrysanthemic acid ethyl ester defined as

$$\frac{Δ [ ( 1 , R , 3 R ) chrysanthemic acid ]}{min * [ proteins in the raw extract ]}$$

ii) the calculation of the % yield of bioconversion of the chrysanthemic ethyl ester substrate in (1R,3R) chrysanthemic acid in experiments conducted at constant pH (pH stat).

This value is calculated by making the ratio between the acid produced (g/L) and the maximum amount of acid obtainable (g/L).

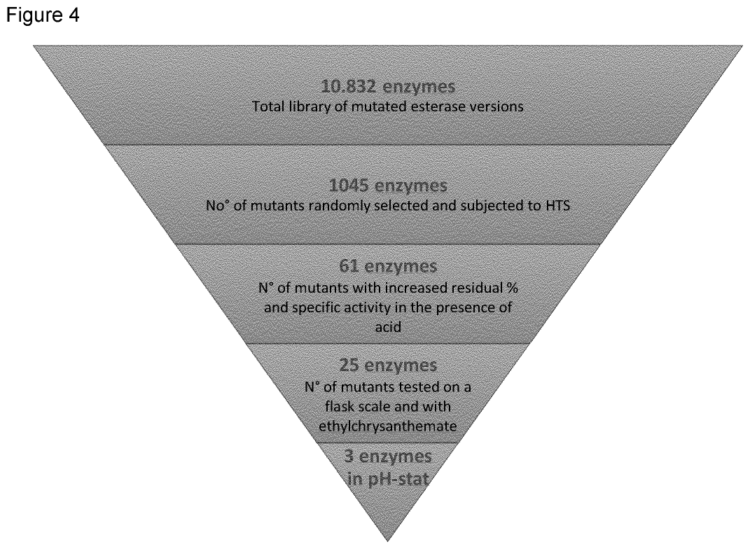

FIG. 4 summarizes the selection scheme carried out and discussed below.

Following the HTS conducted on 1045 samples providing the chromogenic substrate p-nitro-phenylbutyrate, it was found that 61 variants of the starting enzyme S315M show characteristics improved in terms of:
increased residual activity %, or decreased inhibition from the product
increased specific activity (U/mg) in the presence of (1R,3R)-chrysanthemic acid.

In particular, as regards the residual activity %, all 61 selected enzymes have an increase in residual activity compared to the starting enzyme of at least 25%.

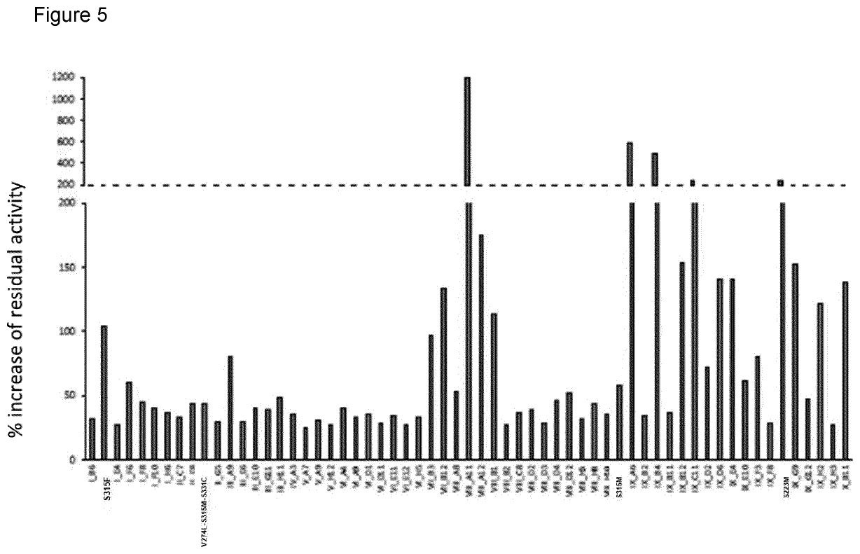

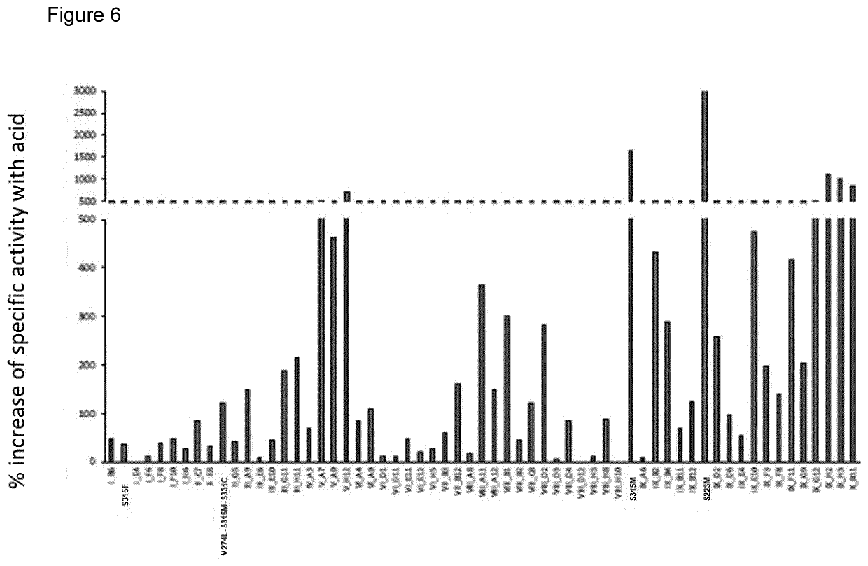

FIGS. 5 and 6 show the % increase in residual and specific activity (in the presence of (1R, 3R)-chrysanthemic acid) of each mutant with respect to the starting enzyme S315M.

Of these 61 enzymatic variants we have selected 25 which have been retested by “scaling up” the procedure.

Specifically, the growth of the 25 E. coli clones expressing the 25 enzymatic variants was performed in 250 mL flasks with 25 mL of self-inducing medium, instead of 1 mL deep wells.

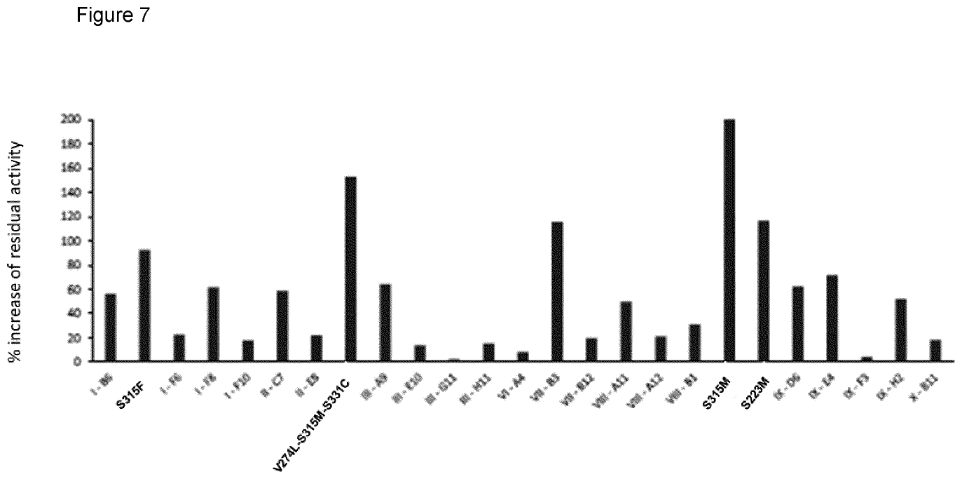

The specific activity in the presence and absence of acid was therefore re-evaluated by providing the substrate chromogenic p-nitro-phenyl-butyrate and the % increase in residual activity was calculated with respect to the starting enzyme S315M (FIG. 7).

All 25 enzyme variants (resulting from flask-scale bacterial growth) exhibit a % increase in residual activity compared to the S315M enzyme, confirming what has been observed in the HTS, albeit with some variation in the absolute values.

The 25 variants were also tested by providing the ethyl ester of chrysanthemic acid substrate.

### Bioconversion of the 3 Selected Enzymatic Variants (V274L-S315M-S331C-S315F-S223M) in pH Stat

The selection of the three comparison mutants with the S315M reference was made from the previous 25 mutants by performing small-scale bioconversions with the chromogenic substrate but in the presence of chrysanthemic acid in the reaction mixture. The mutants with greater activity in the presence of the potential inhibitor (product inhibition) were then selected, thus demonstrating less influence from the acid. Subsequently, performance is naturally estimated with the substrate of interest. Specifically, the reaction in pH stat is carried out in a final volume of 20 mL in the presence of the racemate ethyl ester of chrysanthemic acid as substrate at 10% (v/v). The reaction is carried out at 45° C. for 20 hours during which the pH is maintained at a value of 9.5 by the addition of 1 M NaOH. At the end of the reaction, the amount of (1R, 3R)-chrysanthemic acid is evaluated by HPLC analysis.

### RESULTS AND CONCLUSIONS

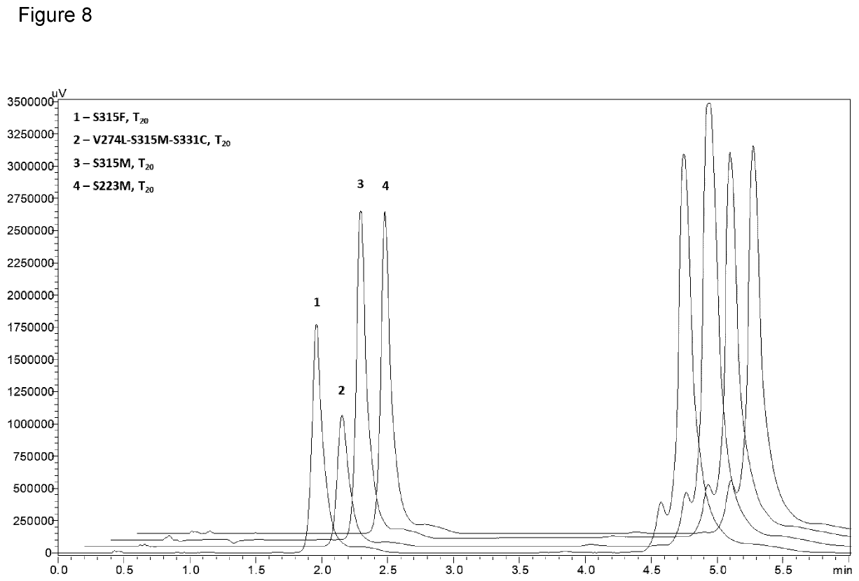

FIG. 8 shows the HPLC chromatograms of the previous bioconversion obtained after 20 hours of reaction (T20) using 10% chrysanthemic acid ethyl ester (v/v). Specifically, the peaks at about 2 min will refer to (1R,3R) chrysanthemic acid while those at about 4.7 min are related to all the chrysanthemic acid ethyl esters, not converted.

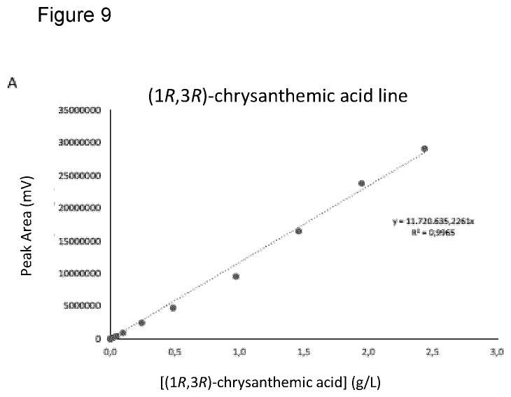

In order to quantify the (1R,3R) chrysanthemic acid produced at the end of the reaction, a calibration line (FIG. 9), with a standard of the aforementioned acid, which correlates the peak area with the known acid concentration, expressed in g/L (Table 1) has been performed. To determine the bioconversion yields, the maximum yield which is theoretically obtainable was first calculated. From the analysis of the starting mixture of isomers of ethyl ester of chrysanthemic acid, the enantiomer ethyl ester of (1R,3R) chrysanthemic acid is present at a concentration of 404.6 g/L. This mixture was used at 10% (v/v) therefore the reaction solution contained 40.46 g/L of ethyl ester of (1R, 3R)-chrysanthemic acid.

Considering the molecular weight of the resulting (1R, 3R)-chrysanthemic acid, the maximum acid concentration obtainable starting from 40.46 g/L of ester is equal to 34.67 g/L. Table 1 shows the data of (1R,3R)-chrysanthemic acid (g/L) obtained and the relative yield % of bioconversion (% referred to the maximum theoretical obtainable yield). In addition, as a control, the previously obtained WT enzyme values are reported.

TABLE 1

| | Area of (1R, 3R)- | Concentration of | |
|---|---|---|---|
| | chrysanthemic | (1R, 3R)-chrysanthemic | Bioconversion |
| Enzyme | acid (mV) | acid (g/L) | Yield % |
| WT | 17058242 | 17.5 | 49.5 |
| S315F | 11573534 | 22.7 | 65.6 |
| S315M | 16298248 | 32.6 | 94.1 |
| S223M | 15276754 | 31 | 89.4 |

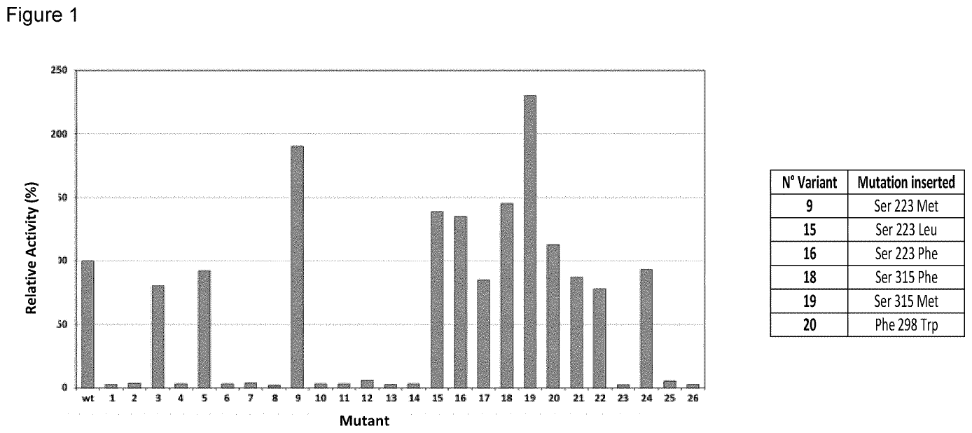

As reported above, the introduction of the point mutation S315M allowed to obtain a better catalyst than the wild-type enzyme, both in terms of specific activity (FIG. 1—mutant 19), and of a lower inhibition by product: the yield of bioconversion of (1R, 3R)-chrysanthemic ethyl ester has in fact increased from 49.5% to 94.1% (Table 1).

Furthermore, as shown in Table 1, the performance of the S223M variant was similar to S315M enzyme; in fact, it transformed a high percentage of substrate after an incubation of 20 hours, with a yield of about 89%, compared to the theoretical maximum obtainable yield.

In conclusion, among the tested mutants, the S315M, S223M, S315F variants have proven to be the optimal ones to catalyze bioconversion of (1R,3R)-ethyl ester of chrysanthemic acid to (1R,3R)-chrysanthemic acid selectively from a mixture of the four possible stereoisomers.

### Example 5. Enzymatic Hydrolysis of Racemate Ethyl Ester of Chrysanthemic Acid 92/8

Ethyl chrysanthemate 92/8 consisting of four stereoisomers was provided to produce (1R, 3R)-chrysanthemic acid by enzymatic hydrolysis reaction with the S315M mutant.

The enzymatic hydrolysis reaction was conducted in batch; in addition to the 92/8 trans/cis ester mixture, glycine, enzyme and aqueous sodium hydroxide were loaded in a reactor, the function of which was to maintain the alkaline environment for the entire duration of the reaction. At the end of the reaction, the enzyme, degraded in an acidic environment by hydrochloric acid and denatured with trichloroacetic acid, was removed by filtration. The remaining solution containing the unconverted esters and the desired (1R,3R) chrysanthemic acid was added with toluene to dissolve the esters and added with a solution (10% w/w) of aqueous sodium hydroxide to bring the chrysanthemic acid into water in the form of sodium salt. From the separation of these two phases, a toluene solution was obtained containing the unconverted esters which were fed to the subsequent workup stage; from the aqueous phase after acidification the product (1R, 3R) chrysanthemic acid was recovered by extraction with toluene and subsequent evaporation of the solvent.

The reaction was carried out on 400 kg of racemate ethyl ester of chrysanthemic acid 92/8 with 16.7 kg of glycine, 444 kg of enzyme dissolved in water, 413 kg of 10% aqueous sodium hydroxide in 3556 kg of water. At the end of the reaction, the raw material was acidified with 116 kg of 37% hydrochloric acid and treated with 11.6 kg of trichloroacetic acid to denature the enzyme and allow it to be filtered. From filtration approximately 17.4 kg of denatured enzyme were obtained and sent for disposal and a solution of toluene and water was introduced for washing the filter and extracting the esters using about 868 kg of it.

The 5808 kg reaction crude was a biphasic system consisting of a toluene solution of the esters and an acid aqueous phase. 3055 kg of acidic waters were discharged from the bottom of the reactor for treatment and 61.8 kg of 50% aqueous sodium hydroxide were added to basify the environment and to bring all the acid into the water as sodium salt.

From the separation of the phases, 1091 kg of toluene solution were obtained and 1724 kg of an aqueous phase which contained salified chrysanthemic acid. 77 kg of 37% (w/w) hydrochloric acid were added to this aqueous phase to result in the desired (1R,3R) chrysanthemic acid and 253 kg of toluene to bring it into the organic phase. After the separation of 1666 kg of water to be sent for disposal, the toluene of the organic phase was evaporated, finally separating 135 kg of (1R,3R) chrysanthemic acid, as unique (1R,3R) form, and with a purity greater than 95%.

From the above description and the above-noted examples, the advantage attained by the product described and obtained according to the present invention are apparent.

1-15. (canceled)

16. An Arthrobacter globiformis esterase mutant having a sequence comprising mutations S315M or S315F of SEQ ID NO: 2, wherein said mutant is capable of asymmetrically hydrolyzing racemic esters of chrysanthemic acid.

17. The Arthrobacter globiformis esterase mutant according to claim 16, having one or two further mutations chosen from the group consisting of:
the double mutant having the mutations S315M and S223M in the amino acid sequence of SEQ ID NO: 2;
the double mutant having the mutations S315F and S223M in the amino acid sequence of SEQ ID NO: 2; and
the triple mutant having the mutations S315M, V274L and S331C in the amino acid sequence of SEQ ID NO: 2.

18. The Arthrobacter globiformis esterase mutant according to claim 16, wherein said mutant has the amino acid sequence of SEQ ID NO: 10, SEQ ID NO: 12, SEQ ID NO: 16, SEQ ID NO: 18 or SEQ ID NO: 20.

19. The Arthrobacter globiformis esterase mutant according to claim 16, wherein said mutant has the amino acid sequence of SEQ ID NO: 10 or SEQ ID NO: 12.

20. The Arthrobacter globiformis esterase mutant according to claim 16, wherein said mutant has the amino acid sequence of SEQ ID NO: 12.

21. The Arthrobacter globiformis esterase mutant according to claim 16, wherein said mutant has the nucleotide sequence of SEQ ID NO: 9, SEQ ID NO: 11, SEQ ID NO: 15, SEQ ID NO: 17 or SEQ ID NO:19.

22. An expression vector including an Arthrobacter globiformis esterase mutant nucleotide sequence chosen from the group consisting of SEQ ID NO: 9, SEQ ID NO: 11, SEQ ID NO: 15, SEQ ID NO: 17 or SEQ ID NO: 19.

23. A transformed host microorganism containing the expression vector according to claim 17, comprising the Arthrobacter globiformis esterase mutant nucleotide sequence.

24. Use of an Arthrobacter globiformis esterase mutant according to claim 16, for the selective preparation of (1R,3R)-chrysanthemic acid or a salt thereof.

25. Use of an Arthrobacter globiformis esterase mutant according to claim 16, for the asymmetrical hydrolysis of cyclopropane racemate derivatives having at least two chiral stereocenters.

26. Use of an Arthrobacter globiformis esterase mutant according to claim 16, for asymmetrical hydrolysis of (C₁-C₆)alkyl ester of chrysanthemic acid, preferably ethyl ester of chrysanthemic acid.
